(19) United States
(12) Patent Application Publication
(10) Pub. No.: US 2024/0197526 A1
(43) Pub. Date: Jun. 20, 2024

(54) GRASPING STRUCTURE FOR OPHTHALMIC SURGERY

(71) Applicant: Alcon Inc., Fribourg (CH)

(72) Inventors: Reto Grüebler, Greifensee (CH); Luca Palmerini, Basel (CH)

(21) Appl. No.: 18/530,996

(22) Filed: Dec. 6, 2023

Related U.S. Application Data
(60) Provisional application No. 63/387,333, filed on Dec. 14, 2022.

(51) Int. Cl. A61F 9/007; A61B 17/29

(52) U.S. Cl. CPC A61F 9/00736; A61B 17/2909

(57) ABSTRACT

An ophthalmic surgical instrument for peeling a retinal membrane includes a handle and an actuator mounted on the handle. A first arm and a second arm extend outwardly from a distal end of an outer tube mounted to the handle. The first arm includes a first pulling surface and a first gripping surface and the second arm includes a second pulling surface and a second gripping surface. The pulling surface of each arm is substantially perpendicular to the gripping surface of each arm and the gripping surfaces of the arms face one another. The first pulling surface and the second pulling surface move toward one another when the outer tube is extended over the first arm and the second arm in order to raise a flap of the retinal membrane that is then gripped between the first gripping surface and the second gripping surface.

### BACKGROUND

The internal limiting membrane (ILM) is a thin transparent membrane positioned between the vitreous and the retina of the eye. The ILM plays a role during the formation of the eye but is not required for the proper function of an adult eye. The ILM may pull at the retina and cause conditions such as macular holes, macular pucker, vitreo-macular traction syndrome, diabetic macular edema, and cystoid macular edema secondary to inflammation or venous occlusive diseases and other conditions. An epiretinal membrane (ERM) is a membrane that may form over the retina in response to damage to the retina, such as due to posterior vitreous detachment.

The ILM or ERM may need to be peeled away from the retina to prevent damage to the retina. Peeling of the ILM or ERM may also be required in preparation for surgical procedures performed on the retina. To peel the ILM or ERM, a surgical instrument is inserted through a cannula (e.g., trocar cannula) within the patient's eye globe. Forceps or a specialized scraper are extended from the instrument and used to raise a flap in the ILM or ERM. The raised flap is then grasped by the forceps and the ILM or ERM is peeled away from the retina using a circular motion. Excess force on the forceps may, however, result in piercing of the retina.

It would, therefore, be an advancement in the art to reduce the risk of retinal damage resulting from ILM or ERM peeling.

### BRIEF SUMMARY

The present disclosure relates generally to a structure for grasping the internal limiting membrane (ILM) or epiretinal membrane (ERM) during ophthalmic surgery.

An ophthalmic surgical instrument for peeling a retinal membrane includes a handle and an actuator mounted on the handle. An outer tube having a proximal end mounted to the handle. A first arm extends outwardly from a distal end of the outer tube and includes a first pulling surface and a first gripping surface substantially perpendicular to the first pulling surface. A second arm extends outwardly from the distal end of the outer tube and includes a second pulling surface and a second gripping surface substantially perpendicular to the second pulling surface. The second gripping surface faces the first gripping surface. The actuator is configured to cause the first arm and the second arm to withdraw into the outer tube thereby causing the first pulling surface and the second pulling surface to move toward one another. As the first pulling surface and the second pulling surface move toward one another, a flap of the retinal membrane is raised between the first gripping surface and the second gripping surface and is gripped between the first gripping surface and the second gripping surface.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 is an isometric view of a surgical instrument having a grasping structure including pulling and gripping surfaces, in accordance with certain embodiments.

FIG. 2A is an isometric view of the grasping structure in an open configuration, in accordance with certain embodiments.

FIG. 2B is an isometric view of the grasping structure in a closed configuration, in accordance with certain embodiments.

FIG. 2C is a cross-sectional view of the grasping structure in a retracted configuration, in accordance with certain embodiments.

FIGS. 3A to 3C are cross-sectional view illustrating an example method of grasping a membrane using the grasping structure, in accordance with certain embodiments.

FIG. 4 is an isometric view showing an ILM being peeled using the grasping structure, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide a surgical instrument including flexible loops for peeling a membrane from a patient's retina. Note that, herein, a distal end of a component refers to the end that is closer to a patient's body while the proximal end of the component refers to the end that is facing away from the patient's body or in proximity to, for example, the handle of the surgical instrument.

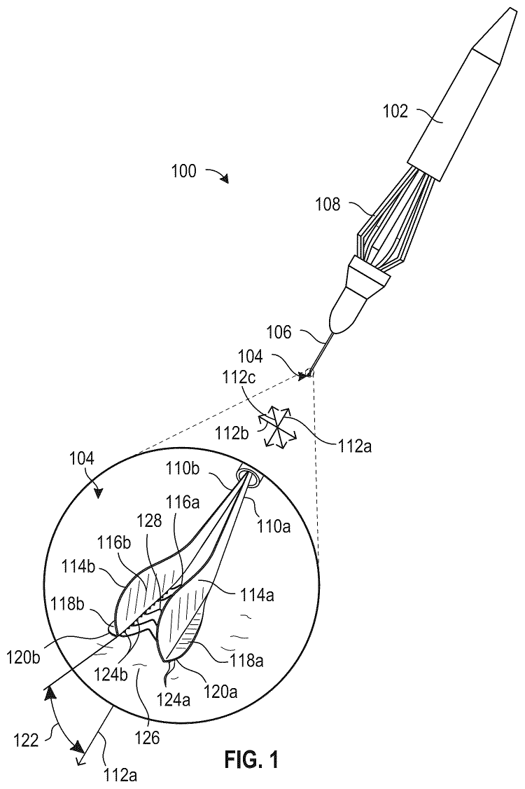

FIG. 1 illustrates a surgical instrument 100, in accordance with certain embodiments, including a handle 102 that is sized and contoured to be grasped by a hand of a surgeon performing an ophthalmic surgical procedure such as peeling of a membrane from a retina of a patient's eye, such as an ILM or ERM. A grasping structure 104 is extendable from a distal end of an outer tube 106 connected to the handle 102. The proximal end of the outer tube 106 is connected to the handle 102. The handle 102 may have a manual control structure mounted thereto. In the embodiment of FIG. 1, the manual control structure includes a deformable basket 108. However, the manual control structure may also be implemented as a slider, button, or any other manual control structure known in the art of ophthalmic surgical instruments.

The outer tube 106 is coupled to the deformable basket 108 and moves outwardly in relation to the handle 102 responsive to compression of the deformable basket 108, and moves inwardly into the handle responsive to decompression of the deformable basket 108. In other embodiments, the deformable basket 108 is coupled to the grasping structure 104 such that the grasping structure is moved relative to the outer tube 106 responsive to compression and decompression of the deformable basket 108.

In FIG. 1, the grasping structure 104 is embodied as arms 110a, 110b that pass through the outer tube 106 and are fixed relative to the handle 102 where the outer tube 106 is actuated by the deformable basket 108. In some embodiments, one or both of the arms 110a, 110b (the arm 110a in the illustrated embodiment) may have a spiral or twisted shape. In other embodiments, neither arm has a spiral or twisted shape (e.g., as shown by the arm 110b). The outer tube 106 defines a longitudinal direction 112a that is parallel to and collinear with an axis of symmetry of the outer tube 106. When extended relative to the outer tube 106, the arms 110a, 110b recoil to the illustrated positions in which the arms 110a, 110b are offset from one another along a transverse direction 112b that is perpendicular to the longitudinal direction 112a. The arms 110a, 110b may be symmetrical in that the arms 110a, 110b are biased outwardly to positions that are substantially (e.g., within 10 percent) the same distance from the axis of symmetry of the outer tube 106. The arms 110a, 110b may be asymmetrical in that the arms recoil to positions such that one arm 110a extends outwardly from the axis of symmetry of the outer tube 106 a greater extent (e.g., at least 50 percent more) than the other arm 110b.

A distal portion of each arm 110a, 110b may include a vertical portion 114a, 114b. The vertical portions 114a, 114b each define inward facing gripping surfaces 116a, 116b that are oriented substantially (e.g., within 10 degrees of) parallel to the longitudinal direction 112a and the vertical direction 112c.

The distal portion of each arm 110a, 110b may also include horizontal portions 118a, 118b secured to edges of the vertical portions 114a, 114b. The horizontal portions 118a, 118b may be implemented as flanges extending outwardly from the vertical portions 114a, 114b, or may be a lower surface of the vertical portion 114a, 114b itself. The horizontal portions 118a, 118b each include downward facing pulling surfaces 120a, 120b that extend outwardly from the gripping surfaces 116a, 116b. The gripping surfaces 116a, 116b may be oriented substantially (e.g., within 10 degrees of) perpendicular to the pulling surfaces 120a, 120b. In certain embodiments, there may be a rounded transition between the pulling surfaces and the gripping surfaces 116a, 116b.

The pulling surfaces 120a, 120b are oriented parallel to a plane that is substantially parallel to the transverse direction 112b and at an angle 122 relative to the longitudinal direction 112a. In certain embodiments, the pulling surfaces 120a, 120b may have barbs 124a, 124b, or similar features, formed thereon. The barbs 124a, 124b may be arrays of barbs forming a structure similar to the scales of shark skin. The barbs 124a, 124b may be pointed inwardly, i.e. barbs 124a pointed toward the pulling surface 120b, and barbs 124b pointed toward the pulling surface 120a. In this manner, the barbs 124a provide greater resistance to relative movement of a membrane 126 away from the pulling surface 120b as compared to movement of the membrane 126 in the opposite direction. Similarly, the barbs 124b provide greater resistance to relative movement of the membrane 126 away from the pulling surface 120a than movement of the membrane 126 in the opposite direction. Accordingly, when the pulling surfaces 120a, 120b are urged toward one another by, for example, extension of the outer tube 106 over the arms 110a, 110b or withdrawal of the arms 110a, 110b into the outer tube 106, the barbs 124a, 124b tend to pull the membrane 126 inwardly, thereby raising a flap 128 that may then be gripped between the gripping surfaces 116a, 116b.

In certain embodiments, the barbs 124a, 124b extend outwardly from the pulling surfaces 120a, 120b by a distance less than the thickness of the membrane 126. For example, the barbs 124a, 124b may extend outwardly from the pulling surfaces 120a, 120b between about 0.8 and about 8 microns, such as between about 1 and about 6 microns, such as between about 2 and about 4 microns.

The pulling surfaces 120a, 120b rest flat, substantially flat, or at least approximately flat on the membrane 126 during use such that the barbs 124a, 124b may grip the membrane 126 and so that a large area of the pulling surfaces 120a, 120b is in contact with the membrane in order to reduce risk of puncturing the membrane 126 and the underlying retina. The angle 122 may encourage bending of the arms 110a, 110b responsive to pressing of the pulling surfaces 120a, 120b against the membrane 126 so that the pulling surfaces 120a, 120b are resting flat on the membrane 126. The angle 122 may be any angle from 0 to 45 degrees.

As is apparent in FIG. 1, the perimeters of the vertical portions 114a, 114b and the horizontal portions 118a, 118b may be rounded to both (a) reduce risk of puncturing a retina and (b) enable the vertical portions 114a, 114b and horizontal portions 118a, 118b to slide smoothly into and out of the outer tube 106 during use.

The grasping structure 104 may be made of a highly flexible material, such as nitinol (a nickel titanium alloy), spring steel, or other surgical-grade material. The high flexibility enables grasping structure 104 to deform elastically when withdrawn into the outer tube 106, thus facilitating greater dimensions of the horizontal portions 118a, 118b, and in certain embodiments, vertical portions 114a, 114b. When extended from the outer tube 106, the horizontal portions 118a, 118b may expand to a width in the transverse direction 112b that is many times the inner diameter of the outer tube 106 and possibly many times the outer diameter of the outer tube, such as 1.5, two, four, or eight times. For example, the height of the gripping surfaces 116a, 116b perpendicular to the vertical direction 112c may be between 0.2 and 0.5 millimeters, and the width of the pulling surfaces 120a, 120b in the transverse direction 112b may be between 0.1 and 0.4 millimeters. The lengths of the gripping surfaces 116a, 116b and the pulling surfaces 120a, 120b in the longitudinal direction 112a may be between 0.2 and 0.8 millimeters.

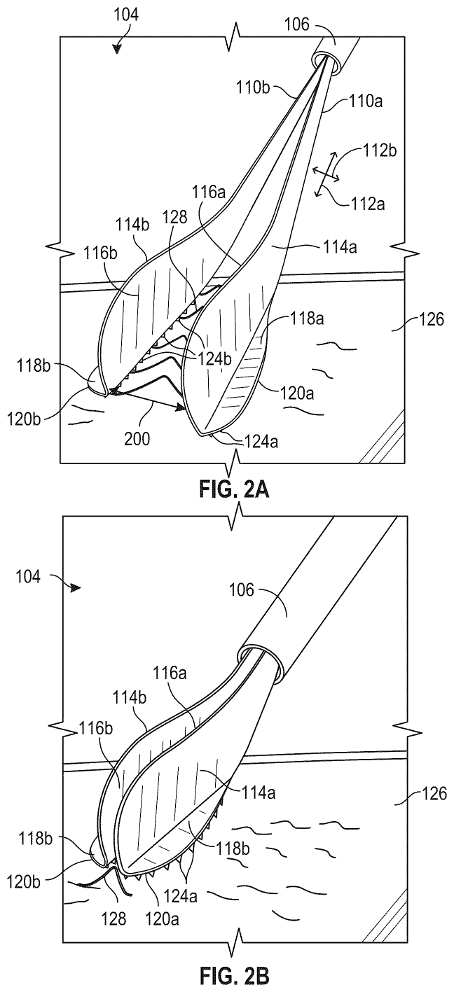

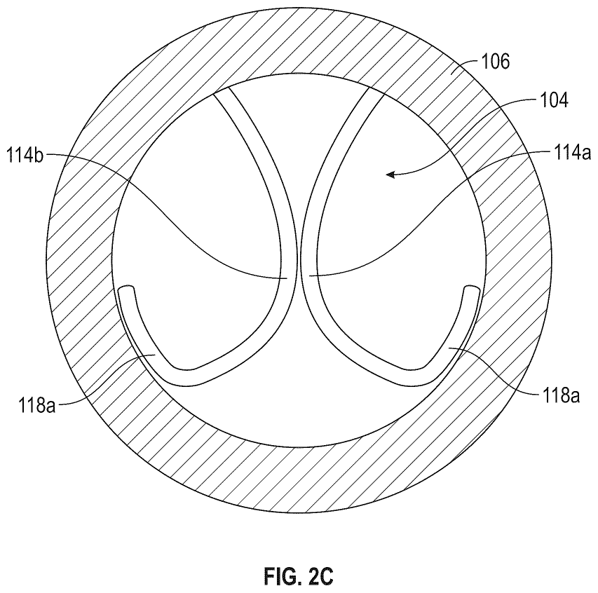

FIGS. 2A to 2C illustrate the grasping structure in an open configuration, a closed configuration, and a retracted configuration, respectively, in accordance with certain embodiments. As shown in FIG. 2A, in use, the outer tube 106 is withdrawn or the arms 110a, 110b are pushed outwardly from the outer tube 106 such that at least a portion of the arms 110a, 110b are extended outwardly relative to the outer tube 106. When extended, the arms 110a, 110b are biased such that gripping surfaces 116a, 116b are separated by a gap 200 in the transverse direction 112b due to recoiling of the arms 110a, 110b. The gap 200 may be comparable in size to the height of a flap 128 to be raised, such as at least 10 times, at least 100 times, or at least 200 times the typical thickness of the membrane 126. For example, the ILM typically has a thickness of 1 and 10 microns.

Referring to FIG. 2B, the outer tube 106 may then be extended or the arms 110a, 110b pulled inwardly into the outer tube 106 thereby drawing the arms 110a, 110b inward relative to the outer tube 106 and possibly parts of one or both of the vertical portions 114a, 114b and horizontal portions 118a, 118b. This forces the pulling surfaces 120a, 120b together such that the barbs 124a, 124b pull on the membrane 126 and raise a flap 128 between the gripping surfaces 116a, 116b. As the arms 110a, 110b are brought closer together, the flap 128 is gripped firmly between the gripping surfaces 116a, 116b. The surgeon may then move the surgical instrument in a circular motion in order to peel a portion of the membrane 126.

Referring to FIG. 2C, when passing through a trocar cannula before and after peeling the membrane 126, the arms 110a, 110b, vertical portions 114a, 114b, and horizontal portions 118a, 118b may be drawn into the outer tube 106. The vertical portions 114a, 114b and horizontal portions 118a, 118b may elastically curl and/or fold in order to fit within the outer tube 106. Alternatively, the vertical portions 114a, 114b and horizontal portions 118a, 118b may be sized to fit within the outer tube 106 without curling or folding or may be sized to fit through the trocar cannula, e.g., not extend outwardly from the outer diameter of the outer tube 106.

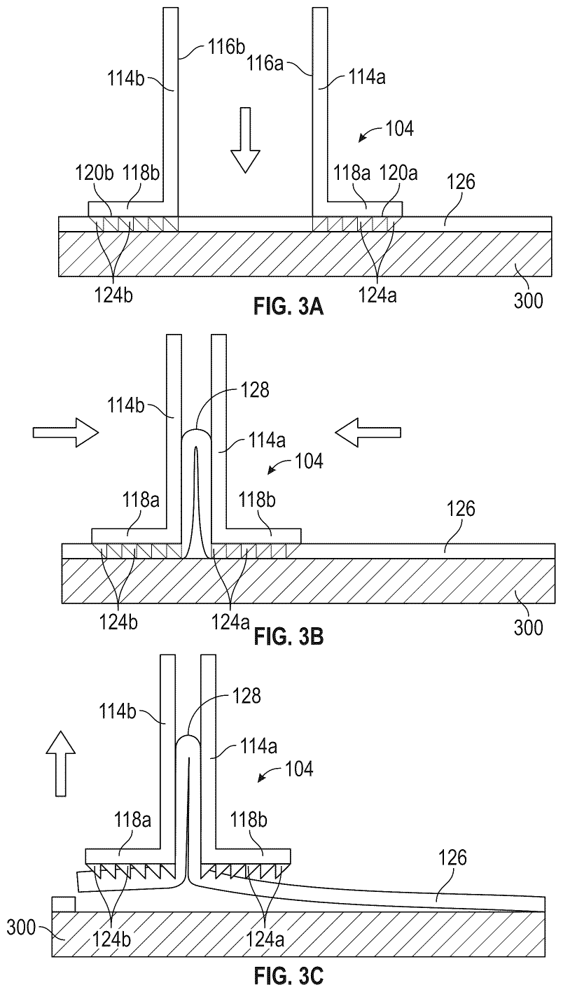

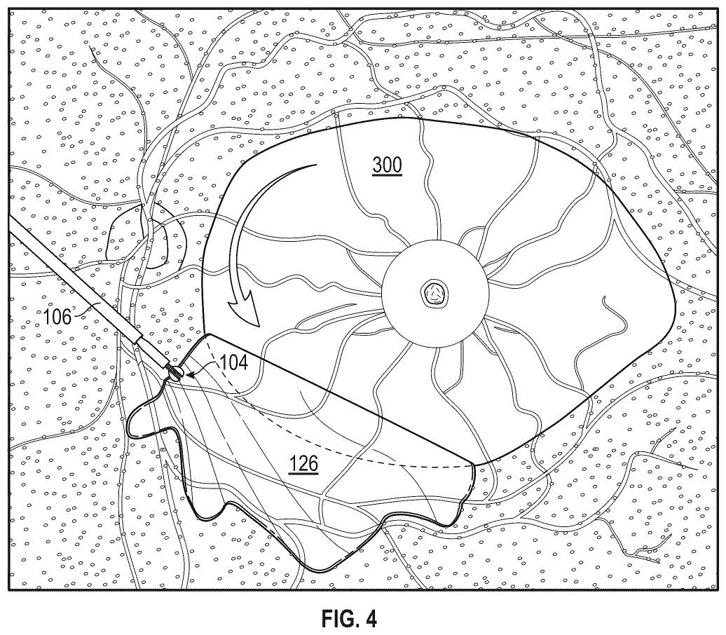

FIGS. 3A to 3C further illustrate the process of peeling the membrane 126 using the grasping structure 104. As shown in FIG. 3A, the pulling surfaces 120a, 120b are placed on the membrane 126 and the barbs 124a, 124b partially penetrate the membrane 126 without penetrating the underlying retina 300. The extent of the barbs 124a, 124b from the pulling surfaces 120a, 120b is preferably less than the thickness of the membrane 126, such as between 0.5 and 0.75 times the thickness of the membrane 126. The outer tube 106 is then extended thereby drawing the pulling surfaces 120a, 120b together as shown in FIG. 3B. FIG. 3B illustrates the case where the arms 110a, 110b are asymmetric such that the pulling surface 120a is moved toward the pulling surface 120b and the pulling surface 120b remains substantially stationary. As the pulling surfaces 120a, 120b are drawn together, a flap 128 of the membrane 126 is raised and is eventually gripped firmly between the gripping surfaces 116a, 116b. As shown in FIG. 3C, the surgeon may then pull the grasping structure 104 away from the retina 300, thereby tearing the membrane 126. As shown in FIG. 4, the surgeon may move the grasping structure 104 in a circular motion to peel a portion of the membrane 126 away from the retina 300.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. An ophthalmic surgical instrument for peeling a retinal membrane, comprising:
a handle;
an actuator mounted on the handle;
an outer tube having a proximal end mounted to the handle;
a first arm extending outwardly relative to a distal end of the outer tube and including a first pulling surface and a first gripping surface substantially perpendicular to the first pulling surface; and
a second arm extending outwardly relative to the distal end of the outer tube and including a second pulling surface and a second gripping surface substantially perpendicular to the second pulling surface, the second gripping surface facing the first gripping surface,
wherein the actuator is configured to cause the first arm and the second arm to withdraw into the outer tube thereby causing the first pulling surface and the second pulling surface to move toward one another for raising a flap of the retinal membrane between the first gripping surface and the second gripping surface, and gripping of the flap between the first gripping surface and the second gripping surface.

2. The ophthalmic surgical instrument of claim 1, wherein the actuator is configured to extend the outer tube in relation to the handle over the first arm and the second arm responsive to movement of the actuator in a first direction.

3. The ophthalmic surgical instrument of claim 2, wherein the actuator is configured to withdraw the outer tube from over the first arm and the second arm responsive to movement of the actuator in a second direction opposite the first direction.

4. The ophthalmic surgical instrument of claim 1, wherein the first arm and the second arm are configured to extend outwardly from an outer diameter of the outer tube when extended outwardly relative to the distal end of the outer tube.

5. The ophthalmic surgical instrument of claim 1, wherein the first arm and the second arm are biased to recoil outwardly when extended from the distal end of the outer tube such that the first arm and the second arm extend outwardly to at least twice a diameter of the outer tube.

6. The ophthalmic surgical instrument of claim 1, wherein the first arm and the second arm are configured to elastically deform sufficiently to fit within the outer tube.

7. The ophthalmic surgical instrument of claim 6, wherein the first arm and the second arm each comprise nitinol.

8. The ophthalmic surgical instrument of claim 1, wherein a first pulling surface includes first barbs configured to grip the retinal membrane and the second pulling surface includes second barbs configured to grip the retinal membrane.

9. The ophthalmic surgical instrument of claim 8, wherein the first barbs and the second barbs have lengths between 0.8 and 8 microns.

10. The ophthalmic surgical instrument of claim 1, wherein the first pulling surface is formed on a first flange extending outwardly from the first gripping surface and the second pulling surface is formed on a second flange extending outwardly from the second gripping surface.

11. A method for peeling a membrane from a retina of a patient's eye, the method comprising:
inserting a distal end of an outer tube through a cannula in the patient's eye;
extending a first arm and a second arm outwardly relative to the distal end of the outer tube;
engaging the membrane with a first pulling surface on the first arm and a second pulling surface on the second arm; and
bringing the first arm and the second arm together such that a flap is raised and grasped between a first gripping surface on the first arm and a second gripping surface on the second arm, the first gripping surface being substantially perpendicular to the first pulling surface and the second gripping surface being substantially perpendicular to the second pulling surface.

12. The method of claim 11, further comprising pulling on the flap effective to peel a portion of the membrane from the retina.

13. The method of claim 11, wherein:
the outer tube is mounted to a handle having an actuator mounted thereto and coupled to the outer tube;
extending the first arm and the second arm from the distal end of the outer tube comprises moving the actuator in a first direction; and
bringing the first arm and the second arm together comprises moving the actuator in a second direction opposite the first direction to draw the first arm and the second arm into the outer tube.

14. The method of claim 11, wherein the first arm and the second arm extend outwardly from an outer diameter of the outer tube when extended from the distal end of the outer tube.

15. The method of claim 11, biasing the first arm and the second arm outwardly from the outer tube when extended from the distal end of the outer tube such that the first arm and the second arm extend outwardly to at least twice a diameter of the outer tube.
